(19) United States
(12) Patent Application Publication
(10) Pub. No.: US 2004/0115174 A1
(43) Pub. Date: Jun. 17, 2004

(54) Angio-immunotherapy

(75) Inventors: Eli Gilboa, Durham, NC (US); Smita Nair, Morrisville, NC (US); David Boczkowski, Morrisville, NC (US)

Correspondence Address: JENKINS & WILSON, PA, 3100 TOWER BLVD, SUITE 1400, DURHAM, NC, 27707, US

(73) Assignee: Duke University

(21) Appl. No.: 10/613,262

(22) Filed: Jul. 3, 2003

Related U.S. Application Data
(60) Provisional application No. 60/393,599, filed on Jul. 5, 2002.

(51) Int. Cl. A61K 39/00

(52) U.S. Cl. 424/93.7; 424/184.1

(57) ABSTRACT

The present invention relates, in general, to cancer therapy and, in particular, to a method of treating cancer that involves immunization against an endothelial-specific product preferentially expressed during tumor angiogenesis or against a factor that contributes to the angiogenic process.

### RELATED APPLICATIONS

This application claims the benefit of U.S. Provisional Patent Application Serial No. 60/393,599, filed Jul. 5, 2002; the disclosure of which is incorporated herein by reference in its entirety.

### TECHNICAL FIELD

The present invention relates to cancer therapy and, in particular, to a method of treating cancer that involves inducing active immunity against an endothelial-specific product preferentially expressed during tumor angiogenesis or against a factor that contributes to the angiogenic process.

### BACKGROUND

Despite the many advances in the field of tumor treatment, cancer remains a major cause of morbidity and mortality. Various active immunotherapy approaches have been developed to induce immune responses to tumor antigens. However, clinical use of these strategies has been limited by challenges such as tolerance to self antigens on tumor cells, the emergence of immunological escape variants and the need to identify potent and broadly expressed antigenic targets.

One approach to immunotherapy that addresses these challenges is the use of antigen presenting cells transfected with tumor RNA. Methods for treating cancers using antigen-presenting cells loaded with RNA are disclosed in U.S. Pat. Nos. 6,306,388 and 5,853,719 and related patents and applications.

Another approach to tumor therapy is the inhibition of angiogenesis. Angiogenesis is the formation of new blood vessels by capillary sprouting from pre-existing vessels. Endothelial cells are normally quiescent and seldom proliferate. in certain physiological processes (e.g., wound healing, hair growth, ovulation and embryogenesis), as well as in pathologic processes (e.g., diabetic retinopathy, psoriasis, atherosclerosis, rheumatoid arthritis, obesity and cancer), proliferation of endothelial cells and neoangiogenesis increase dramatically.

All tumors beyond a minimal size require blood supply and depend on intratumor neoangiogenesis. Increased blood flow to the tumor is necessary for its continued growth. Recent advances in the understanding of the molecular mechanisms underlying the angiogenic process and its regulation have led to the development of anti-angiogenic therapies for the treatment of cancer ^{32, 34}.

Angiostatin and endostatin represent two potent and specific angiogenesis inhibitors that are generated by post translational cleavage from larger precursors, plasminogen and collagen XVIII, respectively. Anti-tumor activity of angiostatin and endostatin has been demonstrated in murine studies. The use of angiogenesis inhibitors in the treatment of angiogenesis-dependent diseases, such as cancer, is described in U.S. Pat. Nos. 5,733,876; 5,854,205; 5,792,845; 6,174,861; 6,544,758 and related patents.

Passive monoclonal antibody-based therapies have also been proposed as a means to inhibit tumor angiogenesis. Monoclonal antibodies specific to various angiogenesis associated antigens, such as vascular endothelial growth factor (VEGF), vascular endothelial growth factor receptor (VEGF-R), and integrins and their use as inhibitors of angiogenesis have been described in U.S. Pat. Nos. 6,524,583; 6,448,077; 6,416,758; 6,365,157 and 6,342,219.

Another intensive area of anti-angiogenic research and development involves the use of small molecular inhibitors ^{32, 34}. These inhibitors are designed to interfere with key pathways that define the angiogenic process.

The leading group of anti-angiogenic agents under development is targeted to matrix metalloproteases (MMPs). Several MMP inhibitors have demonstrated modest clinical benefit as well as undesirable side effects. Small molecule inhibitors which target VEGF and VEGF-R are also under development ²⁴.

Vascular endothelial growth factor (VEGF) and its receptors (VEGFR) play a critical role during angiogenesis and thus they are excellent targets for therapeutic interventions. VEGFR-2 is expressed exclusively in endothelial cells during angiogenesis and is the major transducer of VEGF mediated signals in endothelial cells leading to cell proliferation and migration. The importance of VEGFR-2 signaling for tumor angiogenesis was suggested by the observation that a dominant negative mutant of VEGFR-2 prevented tumor growth in mice ²⁶. VEGFR-2 is up-regulated in tumor-associated endothelial cells but not in the vasculature of the surrounding tissue ²⁷⁻³⁰. In view of the specificity of VEGFR-2 expression in proliferating endothelial cells at sites of angiogenesis and the key role of VEGFR-2 signaling during angiogenesis, interference with VEGFR-2 signaling represents a logical target for the development and clinical testing of anti-angiogenic therapies ³¹⁻³⁴.

Tie2, like VEGFR-2, is a receptor tyrosine kinase upregulated on proliferating endothelial cells and following engagement with its ligand angiopoietin-1, transmits a proangiogenesis signal ^{25,34}. Gene knockout and inhibition studies have shown that Tie2 function is essential during embryogenesis ³⁵ and tumor neoangiogenesis ³⁶⁻³⁸.

VEGF, the ligand for VEGFR-2, is an endothelial-specific growth factor and is essential for angiogenesis ^{24,31}. Targeted inactivation of the VEGF gene in mice causes abnormal blood vessel development and lethality in embryos ^{39,40}. Unlike VEGFR-2 or Tie2, VEGF is expressed in stromal cells during angiogenesis ^{24,31}. VEGF also plays an essential role during tumor angiogenesis as shown by the fact that inhibition of VEGF function suppresses tumor growth in mice ⁴¹. The majority of human and murine tumors induce the expression of VEGF ^{24,31} in response to the progressively hypoxic conditions in the growing tumor ⁴². Indeed, tumors are the main source of VEGF during tumor angiogenesis ^{24,31}. Thus VEGF can serve a dual role as an antigen to target both the tumor and its vasculature.

The Id proteins are a family of four related proteins implicated in the control of differentiation and cell cycle progression. Idl and Id3 are co-expressed temporally and spatially during murine neurogenesis and angiogenesis and are not expressed in the adult normal tissues of murine and human origin. Idl and Id3 are reexpressed in the microvasculature of growing tumors and studies in knockout mice have demonstrated that both Idl and Id3 are required for angiogenesis and vascularization of tumor xenografts. Thus, these molecules present other potential targets for anti-angiogenic therapy.

While current anti-angiogenic therapies for cancer patients have shown some efficacy, the effect is cytostatic rather than cytotoxic. Inhibiting neo-angiogenesis prevents the growth of bulky tumors and may reduce tumor size but it does not eliminate micrometastatic disease. In addition, the use of polypeptide inhibitors presents manufacturing, stability and cost issues. Thus, there remained a need to find more effective tumor therapies that comprise an anti-angiogenic component, alone or in combination with other immunotherapeutic approaches. The present invention addresses that need and provides a novel and effective cancer therapy.

### SUMMARY OF THE INVENTION

The present invention, termed angio-immunotherapy, provides a novel anti-angiogenic composition and method based on active immunization against angiogenesis-related antigens. The term “angiogenesis-related antigen(s)” is used herein to refer to endothelial-specific products that are preferentially expressed during tumor angiogenesis or factors that contribute to the angiogenic process. While the passive administration of specific angiogenesis inhibitors has been described, there have been no previous reports of active immunization against angiogenesis-related antigens.

The present invention further provides a novel therapeutic modality that combines anti-angiogenic therapy and active immunotherapy. The two approaches are compatible therapeutic treatments that provide a synergistic effect.

In one aspect of the invention there is provided a composition for the treatment or prevention of cancer. The composition comprises a plurality of antigen presenting cells transfected with nucleic acid encoding at least one angiogenesis-related antigen.

The antigen presenting cells are preferably dendritic cells and the angiogenesis-related antigen is preferably selected from the group consisting of Id1, VEGFR-2, Tie2 and VEGF.

In a particularly preferred embodiment, dendritic cells are further transfected with nucleic acid encoding at least one tumor antigen. The nucleic acid may be total mRNA from tumor cells or synthetic mRNA encoding a selected tumor-associated antigen.

In another aspect of the invention, a method for the prevention or treatment of cancer is provided. The method comprises obtaining antigen presenting cells from a patient in need of therapy, introducing into those antigen presenting cells in vitro RNA encoding an angiogenesis-related antigen, thereby producing RNA loaded antigen presenting cells, and administering the RNA loaded antigen presenting cells to the patient.

In a preferred embodiment, RNA encoding a tumor antigen is also introduced into the antigen presenting cells thereby producing RNA loaded antigen presenting cells which are capable of presenting both angiogenesis-related antigen and tumor antigen. The tumor RNA and the angiogenesis-related RNA may be introduced at the same time or sequentially.

In another aspect of the invention, RNA loaded antigen presenting cells are prepared as described above. The RNA loaded antigen presenting cells are then contacted with T lymphocytes to generate immune cells in vitro. The in vitro generated CTL are then administered to the patient. As used herein the term “immune cells” refers to cytotoxic T cells, helper T cells, B cells, NK cells and other immune modulating cells.

Objects and advantages of the present invention will be clear from the description that follows.

### BRIEF DESCRIPTION OF THE DRAWINGS

FIG. 1 is a graph demonstrating the inhibition of lung metastases in mice immunized with DC transfected with Idl mRNA;

FIG. 2 is a graph illustrating the effect on lung weight of co-immunization with Idl and B16 tumor RNA transfected DC;

FIG. 3 is a graph illustrating the induction of CTL activity in mice immunized with dendritic cells transfected with VEGF and VEGFR-2 mRNA;

FIG. 4 illustrates the inhibition of angiogenesis in mice immunized against angiogenesis-associated products;

FIG. 5A illustrates inhibition of tumor growth after immunization with DC transfected with VEGF, VEGFR-2 and Tie2 mRNA in a melanoma model;

FIG. 5B illustrates inhibition of tumor growth after immunization with DC transfected with VEGF, VEGFR-2 and Tie2 mRNA in a bladder tumor model;

FIG. 6A shows the results of combination therapy with B16 tumor antigens and Tie2;

FIG. 6B illustrates the results of combination therapy with MBT-2 mRNA or TERT MRNA and VEGF or VEGFR-2;

FIG. 6C illustrates the time to appearance of palpable tumors;

FIG. 7A Illustrates the results of immunotherapy of tumor bearing mice with DC transfected with angiogenesis-associated and tumor antigens as indicated by tumor size at 18 days post-transplantation;

FIG. 7B Illustrates the results of immunotherapy of tumor bearing mice with DC transfected with angiogenesis-associated and tumor antigens as indicated by tumor size at 25 days post-transplantation;

FIG. 7C illustrates the time to appearance of palpable tumors in mice receiving a combination of VEGFR-2 and TRP-2;

FIG. 7D illustrates the time to appearance of palpable tumors in mice receiving a combination of VEGF and TRP-2;

FIG. 8A illustrates the effect of immunization with VEGFR-2 mRNA transfected DC on fertility in mice at one week after the final immunization; and

FIG. 8B illustrates the effect of immunization with VEGFR-2 mRNA transfected DC on fertility in mice at eight weeks after the final immunization.

### DETAILED DESCRIPTION OF THE INVENTION

The present invention relates to a method of treating cancer that comprises inducing active immunity in a patient in need of such treatment against: i) an endothelial-specific product that is preferentially expressed during tumor angiogenesis, and/or ii) a factor that contributes to the angiogenic process. These endothelial-specific products and factor are jointly referred to herein as “angiogenesis-related antigens”. The present invention takes advantage of the facts that i) an immune response can be stimulated against a normal gene product that is preferentially, although not necessarily exclusively, expressed in tumor microvasculature, ii) such a response inhibits tumor progression, and iii) significant toxicity (autoimmunity) does not result. The therapeutic approach of the present invention can be used in combination with other modalities for treating cancer such as radiation, chemotherapy and conventional immunotherapy.

In accordance with the present invention, active immunity can be induced using a variety of approaches. For example, angiogenesis-related antigens can be administered directly either as a composition (vaccine composition) comprising a single antigen type or as a composition comprising a mixture of different types of angiogenesis-related antigens. The antigens used can be produced chemically or recombinantly or the antigens can be isolated from natural sources.

Active immunity can also be induced in accordance with the invention by administering nucleic acid (RNA or DNA) encoding one or more angiogenesis-related antigen. The nucleic acid can be incorporated into a vector (e.g., a viral vector, such as an adenoviral vector, an adenoassociated viral vector, or a vaccinia viral vector). Alternatively, the nucleic acid can be administered in association with a transfection facilitating agent, such as a liposome. Further, the nucleic acid (e.g., DNA present in plasmid) can be administered as naked nucleic acid (see, for example, U.S. Pat. No. 5,589,466) or it can be administered using a gene gun (that is, coated on a particle, such as a gold bead).

In a preferred embodiment, induction of active immunity is effected by administering to the patient antigen presenting cells (APCs) loaded with an angiogenesis-related antigen or transfected in vitro with nucleic acid (DNA or RNA) encoding at least one angiogenesis-related antigen.

Nucleic acid transfection can be effected using conventional techniques well known to those skilled in the art, such as lipid-mediated transfection, electroporation and calcium phosphate transfection. Peptide pulsing of APCs can be effected using art recognized methodologies. (See, for example, U.S. Pat. No. 5,853,719.)

Advantageously, the APCs are professional APCs, such as dendritic cells or macrophages. However, any APC can be used (e.g., endothelial cells or artificially generated APCs). While it is preferred that the cells administered to the patient be derived from that patient (autologous), APCs can be obtained from a matched donor or from a culture of cells grown in vitro. Methods for matching halopytes are known in the art.

The use of RNA-transfected APC's in the method of the invention is particularly advantageous, for example, over the use of protein/peptide pulsed APC's, for reasons that include ease of antigen generation. Given a sequence, the corresponding mRNA can be generated using, for example, RT-PCR and transcription, with cloning of the cDNA intermediate into bacterial plasmid an option but not prerequisite ^{12, 13}. The need to manufacture protein antigen or to identify class I and class II peptides corresponding to specific MHC alleles can thus be avoided.

Angiogenesis-related antigen-encoding nucleic acid for use in the invention can be isolated from natural sources (amplified as necessary) or synthesized chemically or recombinantly using conventional techniques.

Angiogenesis-related antigens suitable for use in the present invention include fetal or embryonic gene products re-expressed in tumor microvasculature (e.g., Id-1 and Id-2), VEGF receptors upregulated in the tumor microvasculature (e.g., VEGFR-2), and the endothelial specific product Tie-2. Angiopoietin-1 is another antigen that is useful in the present invention.

VEGF is expressed by the tumor stroma, the tumor itself or both. Therefore, VEGF is a prototype antigen that can elicit a dual immune response against both the tumor vasculature and the tumor or tumor stroma. Immunotherapy using VEGF, Id-1 and VEGF/Id-1 prototype antigens (or nucleic acids encoding same) can be particularly advantageous.

In accordance with the invention, active immunity can be induced against angiogenesis-related antigens alone or in combination with tumor antigens (e.g., TERT or total tumor derived antigenic mixture) (see U.S. Pat. No. 5,853,719).

The invention can be used to treat an existing tumor or prevent tumor formation in a patient (a human or non-human animal) (e.g., melanoma tumors, bladder tumors, breast cancer tumors, colon cancer tumors, prostate cancer tumors, and ovarian cancer tumors). It is preferable that treatment begin before or at the onset of tumor formation, and continue until the cancer is ameliorated. However, the invention is suitable for use even after a tumor has formed. In treating a patient in accordance to the invention, the optimal dosage depends on factors such as the weight of the patient, the severity of the cancer, and the nature of the antigen targeted.

When APCs are used, the dosage of cells is based on the body weight. Typically, a dosage of 10⁵ to 10⁸ cells/kg body weight, preferably 10⁶ to 10⁷ cells/kg body weight can be administered in a pharmaceutically acceptable excipient to the patient. The cells can be administered by using infusion techniques that are commonly used in cancer therapy. The optimal dosage and treatment regime for a particular patient can readily be determined by one skilled in the art by monitoring the patient for signs of disease and adjusting the treatment accordingly. The treatment can also include administration of mitogens (e.g., phyto-hemagglutinin) or lymphokines (e.g., IL-2 or IL-4) to enhance T cell proliferation.

The present invention demonstrates that combination of anti-angiogenic therapy and tumor immunotherapy of cancer is synergistic. Inhibition of angiogenesis by active immunotherapy to control tumor growth offers several attractive features. Firstly, active immunotherapy can induce a state of reduced angiogenic activity. Second, immunotherapy, like other anti-angiogenic strategies, provides multiple common targets, “universal” antigens, to inhibit tumor angiogenesis. In addition, due to the genetically stable nature and limited proliferative capacity of endothelial and stromal cells, emergence of antigen-loss or antigen processing-loss variants is significantly reduced compared to that of tumor cells. Furthermore, an especially attractive feature of anti-angiogenic immunotherapy is that it can be combined with tumor immunotherapy to deliver two distinct and potentially synergistic treatment modalities using a common procedure, immunization.

Immunization with mRNA-transfected DC is emerging as an efficient strategy to stimulate cellular immunity, and the present invention extends the use of this approach to angiogenesis-associated targets. A particularly useful feature of using mRNA-encoded antigens is the ease of isolating and generating mRNAs. cDNAs can be isolated from cells expressing the desired antigen by simple RT-PCR techniques and mRNA can be generated in pure form and in large quantities using cell-free enzymatic reactions. In the examples below, mRNA technology was used to study three angiogenic targets, VEGFR-2, Tie2 and VEGF. It is readily apparent that the list can be readily expanded with candidates provided by the genomic revolution. Another advantage of the present invention is that the generation of mRNA-encoded antigens is comparatively simple and inexpensive, and the regulatory requirements are straightforward.

The feasibility of the present invention has been demonstrated in several experiments. While specific antigen and protocols have been used, it is clearly apparent that the invention can encompass other antigens and different assay methods.

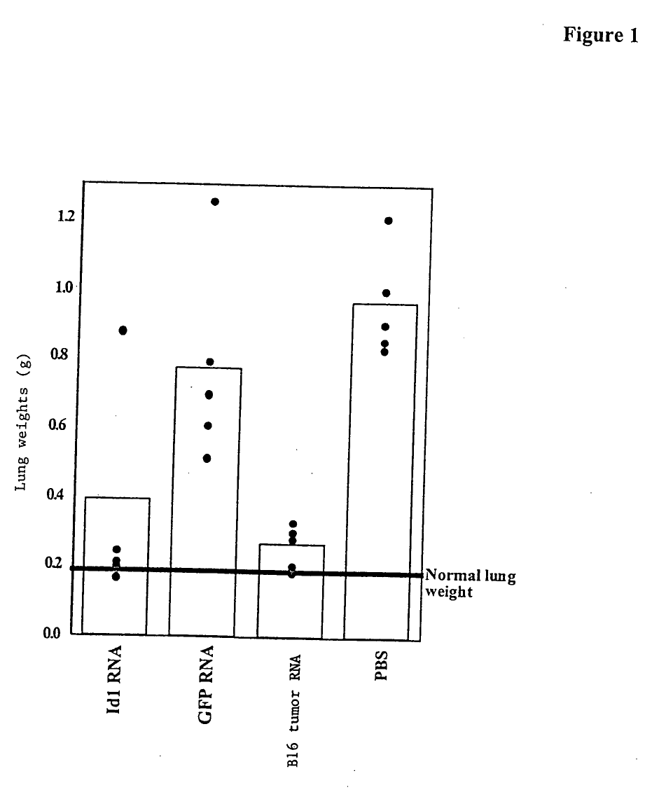

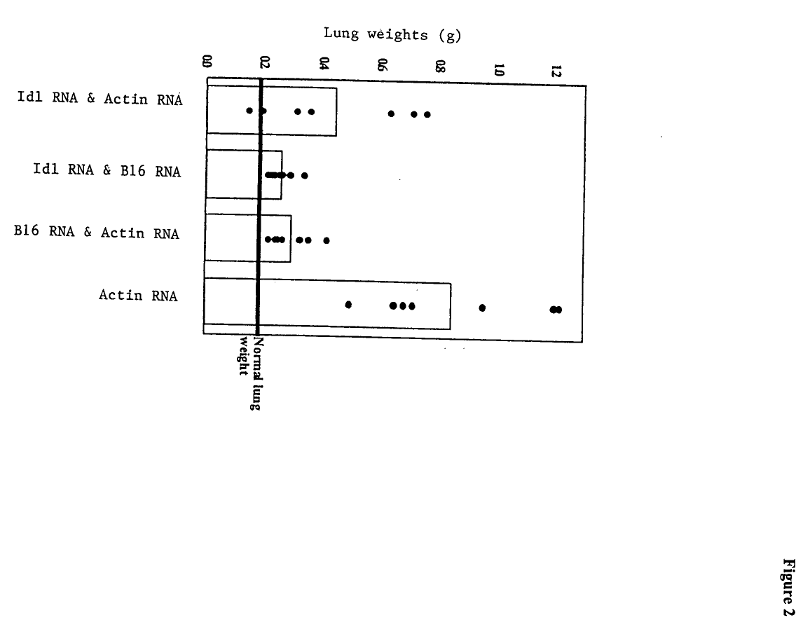

As shown in FIG. 1 and discussed further in Example 4, immunization with Id1 RNA transfected dendritic cells resulted in a significant reduction in lung metastases as compared to control animals. FIG. 2 and example 5 illustrate that this anti-tumor effect can be augmented by co-immunization with Id1 RNA and B16 (tumor) RNA transfected dendritic cells.

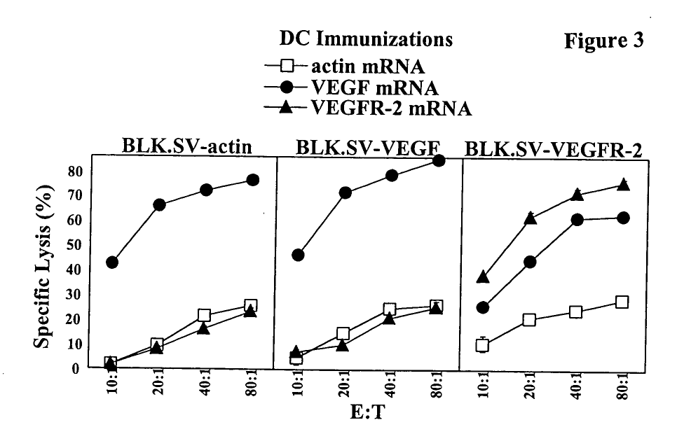

As demonstrated in FIG. 3 and discussed further in Examples 7 and 8, induction of CTL responses against VEGF and VEGFR-2 shows that it is possible to break tolerance against angiogenesis-associated targets. This leads to reduced angiogenic activity in the immunized animals as shown in FIG. 4 and discussed in Examples 9 and 10. These results indicate that induction of immune response against VEGF and VEGFR-2 has potent anti-angiogenic effects.

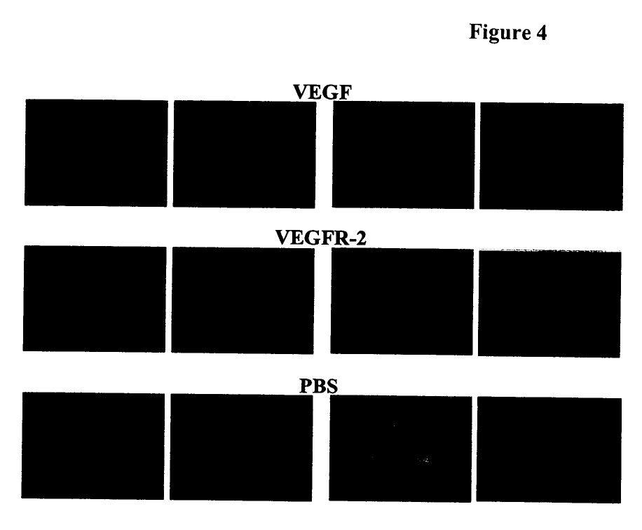

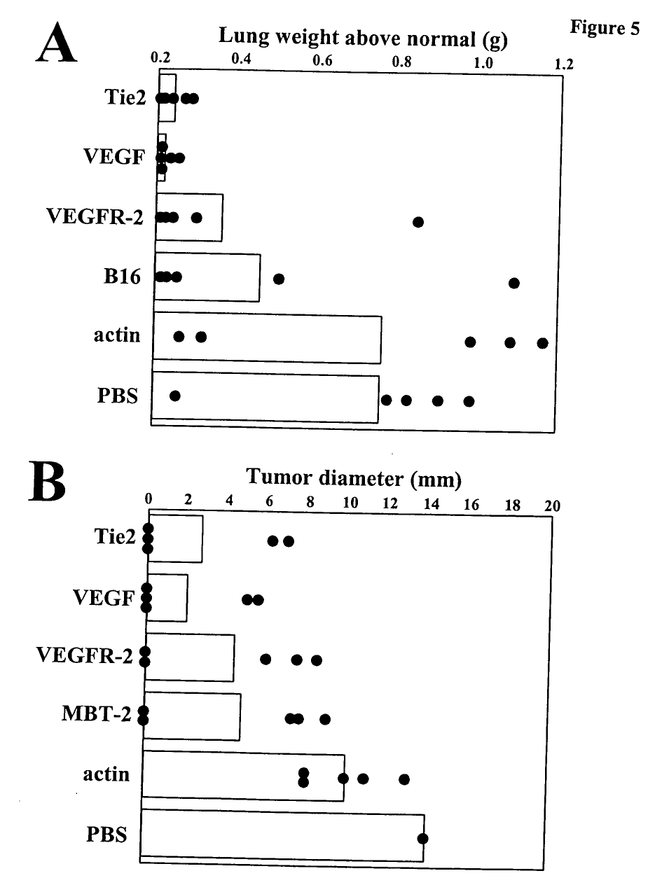

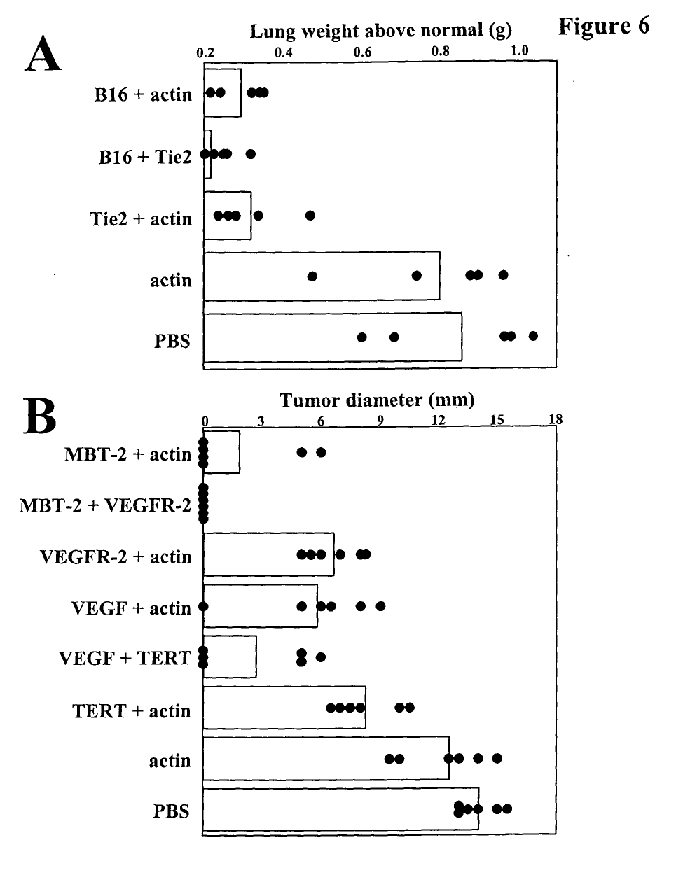

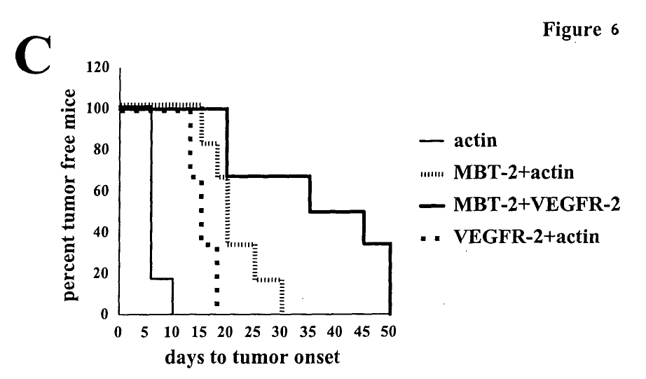

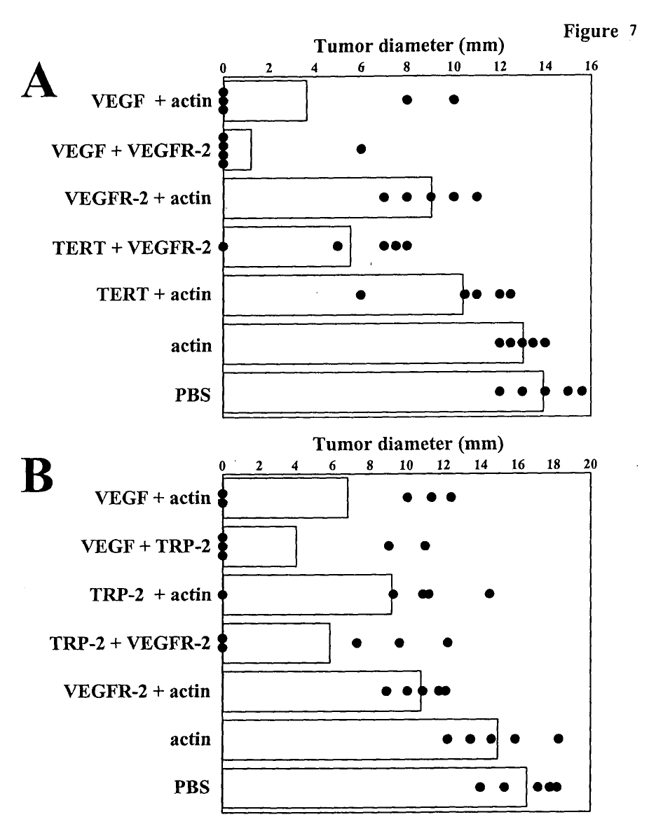

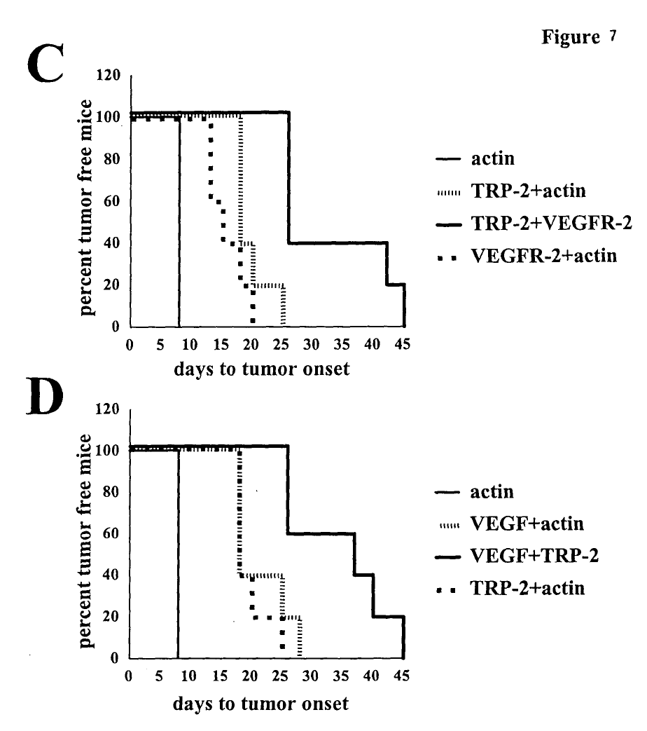

Immunization against the angiogenesis-associated products VEGFR-2, Tie2 or VEGF was accompanied by inhibition of tumor growth in the B16/F10.9 melanoma metastasis and the MBT-2 bladder cancer models. Tumor inhibition was seen when mice were immunized before tumor challenge as shown in FIGS. 5 and 6 and discussed further in Examples 11 and 12. Tumor inhibition was also seen in the setting of pre-existing tumor burden, as discussed in Examples 13 and 14 and shown in FIG. 7. Since VEGFR-2 or Tie2 are expressed in proliferating endothelial cells, but not in MBT-2 or B16/F10.9 tumor cells, the observed tumor inhibition was an indirect consequence of interfering with the tumor neovascularization process. This conclusion is consistent with the observation that immunization against VEGFR-2 is accompanied by a reduced state of angiogenesis in the immunized animal. Unlike VEGFR-2 or Tie2, VEGF is expressed by stromal cells and tumor cells, including the B16/F10.9 and MBT-2 tumor cells used in this study. Thus, VEGF immunization may mediate its anti-tumor effect via inhibition of angiogenesis or direct antitumor immunity.

The experiments shown in FIGS. 6 and 7 establish the potential value of combining anti-angiogenic therapy and tumor immunotherapy. Immunization with syngeneic tumor RNA (B16/F10.9 or MBT-2) stimulates tumor-specific non-crossreactive protective immunity and thereby targets the tumor directly whereas immunization with VEGFR-2 or Tie2 mRNA targets the tumor vascularization process. As shown in FIG. 6, mice immunized with both syngeneic tumor RNA and endothelial specific mRNA (VEGFR-2 or Tie2) exhibited a superior antitumor effect compared to mice immunized with either RNA alone. Furthermore, in the setting of pre-existing disease, co-immunization against tumor (TERT or TRP-2) and angiogenesis-specific (VEGFR-2) targets exhibited a pronounced inhibitory effect on tumor growth (FIG. 5). These experiments also illustrate another key feature of inhibiting angiogenesis via active immunotherapy, namely the ability to deliver two compatible and synergistic cancer treatment modalities by a single protocol, immunization. Combination immunotherapy against VEGF and either TERT (FIG. 6B), VEGFR-2 (FIG. 7A) or TRP-2 (FIGS. 7B and 7D) was also synergistic, underscoring the value of targeting two defined and broadly expressed (“universal”) antigens. Although, in this instance it was not clear whether the contribution of VEGF was inhibition of angiogenesis, direct antitumor immunity or a combination of both.

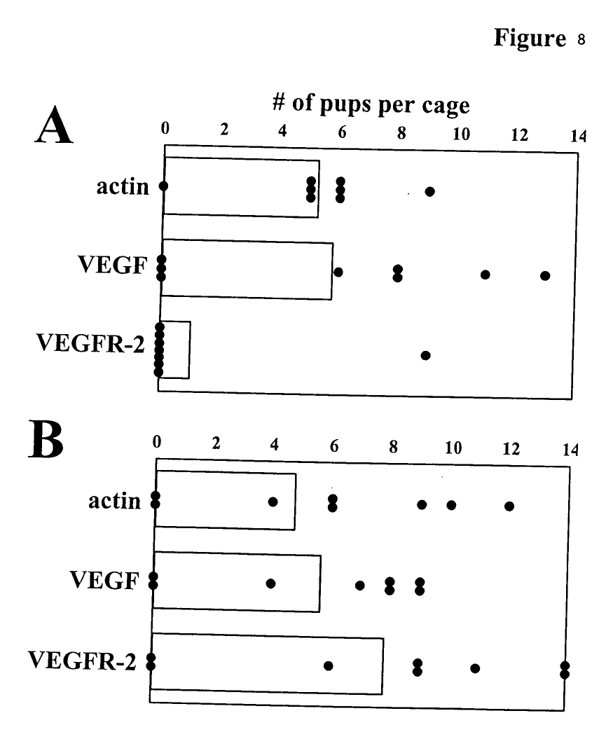

A primary concern of immunizing against angiogenesis-associated products is interference with normal angiogenesis, especially if the effect is sustained. No significant adverse effects were seen in mice immunized against angiogenesis-associated products in this and previous studies under conditions that significant antitumor effects were seen. As shown in FIG. 8, no signs of morbidity or mortality were seen in the immunized animals except for a transient impairment of fertility in mice immunized against VEGFR-2, but not VEGF. These observations are consistent with previous studies which have shown that anti-angiogenic therapy exhibits differential susceptibility on tumor growth and wound healing 48,49, suggesting that a partial and transient reduction in angiogenic activity could suffice to impact on tumor growth without eliciting serious adverse effects. Furthermore, since functional immunological memory will require repeated immunizations 50,51, the persistence of an active anti-angiogenic immune response can be controlled simply by terminating vaccination.

The results described above demonstrate that anti-angiogenic immunotherapy is an effective anti-tumor modality. The effects seen with active immunization against angiogenesis related antigens can be augments by combination with active immunization against tumor antigens. While specific antigens and protocols have been referred to herein, it is clearly apparent that other angiogenesis related antigens and tumor antigens will have similar effects.

The above disclosure generally describes the present invention. It is believed that one of ordinary skill in the art can, using the preceding description, make and use the compositions and practice the methods of the present invention. A more complete understanding can be obtained by reference to the following specific examples. These Examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art. All journal articles and other documents such as patents or patent applications referred to herein are hereby incorporated by reference.

### EXAMPLES

Although specific terms have been used in these examples, such terms are intended in a descriptive sense and not for purposes of limitation. Methods of molecular biology, cell biology and immunology referred to but not explicitly described in the disclosure and these examples are reported in the scientific literature and are well known to those skilled in the art.

### Example 1

Mice and Murine Cell Lines

Mice. 4-6 weeks old C57BL/6 mice (H-2b) and C3H/HeN mice (H-2k) were obtained from the Jackson Laboratory, Bar Harbor, Me. In conducting the research described in this paper, the investigators adhered to the “Guide for the Care and Use of Laboratory Animals” as proposed by the committee on care of Laboratory Animal Resources Commission on Life Sciences, National Research Council. The facilities at the Duke vivarium are fully accredited by the American Association for Accreditation of Laboratory Animal Care.

Cell lines. The F10.9 clone of the B16 melanoma of C57BL/6 origin is a highly metastatic, poorly immunogenic and a low class I expressing cell line 16. EL4 is a thymoma cell line (C57BL/6, H-2b). The murine MBT-2 cell line, derived from a carcinogen-induced bladder tumor in C3H mice 17, was obtained from Dr. T. Ratliff (Washington University, St. Louis, Mo.). The SV40-transformed B6 fibroblast cell line, BLK.SV (TIB-88), was obtained from ATCC. Cells were maintained in DMEM supplemented with 10% fetal calf serum (FCS), 25 mM HEPES, 2 mM L-glutamine and 1 mM sodium pyruvate. Murine precursor-derived DC were generated in the presence of GM-CSF supernatant harvested from F10.9 cells transfected with the GM-CSF cDNA. Actively growing F10.9/GM-CSF cells were cultured in RPMI 1640 supplemented with 5% FCS, 1 mM Na pyruvate, 0.1 mM non-essential amino acids, 100 IU/ml penicillin, 100 μg/ml streptomycin and 5×10-5 M β-mercaptoethanol and 10 mM HEPES (complete RPMI) at 37° C. and 5% CO2. GM-CSF-containing supernatant was harvested after 24 h of capillary culture. The GM-CSF supernatant was used to generate murine DC at a final dilution of 0.1%. The concentration of GM-CSF used was determined by ELISA.

### Example 2

Preparation Of RNA Transfected Dendritic Cells

BMDC (bone marrow precursor-derived dendritic cells) were generated from bone marrow progenitors as previously described 18. Briefly, marrow from tibias and femurs of C57BL/6 mice were harvested followed by treatment of the precursors with ammonium chloride Tris buffer for 3 min at 37° C. to deplete the red blood cells. The precursors were plated in RPMI-5% FCS with GM-CSF (15 ng/ml) and IL-4 (10 ng/ml, Peprotech (Rocky Hill, N.J.). Cells were plated at 106/ml and incubated at 37° C. and 5% CO2. 3 days later the floating cells (mostly granulocytes) were removed and the adherent cells replenished with fresh GM-CSF and IL-4 containing medium. 4 days later the non-adherent cells were harvested (immature day 7 DC), washed and replated at 106/ml in GM-CSF and IL-4 containing medium. After 1 day the non-adherent cells were harvested, washed and electroporated with RNA.

Total RNA was isolated from actively growing tumor cell lines using the RNeasy kits (Qiagen) following the manufacturers protocols.

Electroporation was performed as previously described for human DC 19,20, with small modifications. Briefly, DC were harvested on day 8, washed and gently resuspended in Opti-MEM (GIBCO, Grand Island, N.Y.) at 2.5×107/ml. The used DC culture media was saved as conditioned media for later use. Cells were electroporated in 2 mm cuvettes (200 μl of DC (5×106 cells) at 300 V for 500 μs using an Electro Square Porator ECM 830, BTX, San Diego, Calif.). The amount of IVT RNA used was 2 μg and total tumor RNA was 10 μg, per 106 DC. Cells were immediately transferred to 60 mm tissue culture petridishes containing a 1:1 combination of conditioned DC growth media and fresh RPMI-5% FCS with GM-CSF and IL-4. Transfected cells were incubated at 37° C., 5% CO2 overnight, washed two times in PBS and then injected into mice.

### Example 3

Tumor Challenge Models

B16/F10.9 melanoma model: DC were transfected with the various RNA preparations and naive, syngeneic mice were immunized intravenously with 5×105 precursor-derived DC per mouse in 200 μl PBS, three times at 7-day intervals. Mice were challenged with 5×104 F10.9 cells intravenously 8-10 days after the final immunization. Mice were sacrificed based on the metastatic death in the control groups. Metastatic loads were assayed by weighing the lungs.

MBT-2 murine bladder tumor model: DC were transfected with the various RNA preparations and naive, syngeneic mice were immunized intravenously with 5×105 precursor-derived DC per mouse in 200 μl PBS, three times at 7-day intervals. Mice were challenged with 2-5×105 MBT-2 cells subcutaneously (in the flank) 8-10 days after the final immunization. Tumor growth was evaluated every other day starting on day 6. Mice were sacrificed once the tumor size reached 20 mm.

For experiments testing synergy between different antigens, mice were immunized two times with 3×105 DC in 100 μl for each antigen for a combined 6×105 DC in 200 μl per mouse.

### Example 4

Immunization Against Id-1

Induction of protective anti-tumor immunity by immunization against Idl was tested in the B16 melanoma experimental metastasis system. As described above, mice were first immunized and then challenged intravenously with B16 melanoma tumor cells (highly metastatic clone, F10.9 is used). 28 days later, the mice were sacrificed and the metastatic load in the lung was determined by weighing the lungs. As shown in FIG. 1, immunization with B16 tumor RNA transfected DC causes a significant reduction in lung metastasis in this model. Immunization with Idl RNA transfected DC also leads to a lower metastatic load.

### Example 5

Combination Therapy with Idl and B16 RNA

To determine whether anti-Idl and anti-tumor immunotherapy are synergistic, the same experimental protocol as described above was used with the exception that the intensity of immunization was reduced to two cycles from three cycles to better observe a difference between vaccination with tumor RNA and Idl +tumor RNA.

As shown in FIG. 2, immunization with either Idl or B16 RNA transfected DC inhibited lung metastasis in a significant manner confirming the results shown in FIG. 1. Combination of Id1+B16 RNA vaccination is more potent, albeit not in a statistically significant manner. This may be because, in this particular experiment, two immunizations with tumor RNA resulted in a very significant reduction in tumor metastasis largely obscuring a potential additive effect of co-immunization with Id1.

### Example 6

Preparation of VEGF, VEGFR-2, Tie2, TRP-2, Telomerase and Actin RNA

Creation of pSP73-Sph/A64. Oligonucleotides containing 64 A-T bp followed by an Spe I restriction site were placed between the EcoR I and Nar I sites of pGEM4Z (Promega) to create the plasmid pGEM4Z/A64. The Hind III-Nde I fragment of pGEM4Z/A64 was cloned into pSP73 (Promega) digested with Hind III and Nde I to create pSP73/A64. The plasmid pSP73-Sph was created by digesting pSP73/A64 with Sph I, filling in the ends with T4 DNA polymerase and re-ligating. pSP73-Sph/A64/Not contains a Not I restriction site adjacent to the Spe I site. C. Kontos (Duke University Medical Center, Durham, N.C.) generously provided plasmids containing murine VEGF, VEGFR-2 and Tie2. The cDNAs were amplified with Advantage DNA polymerase (Clontech) for cloning into pSP73-Sph.

Cloning of SP73-Sph/VEGF/A64. The forward primer 5′TATATATCTAGAGCCACCATGGCACCCACGACAGAAGGAGAGCAGAAG -3′ (SEQ ID NO: 1) and reverse primer 5′-TATATAGAATTCTCACCGCCTTGGCTTGTCACATC-3′ (SEQ ID NO: 2) were used to amplify a truncated version of the VEGF coding region, not including the signal sequence, from the plasmid and was cloned into the Xba I-EcoR I sites of pSP73-Sph/A64.

Cloning of pSP73-Sph/VEGFR-2/A64. VEGFR-2 was amplified in three reactions using the following primers: For bases 1-1420, 5′-TATATACTCGAGGCCACCATGGAGAGCAAGGCGATGC TAGCTG-3′ (SEQ ID NO: 3) and 5′-ATTAATCTAGACTAGTTGGACTC AATGGGGCCTTC-3′ (SEQ ID NO: 4). For bases 1420-2730, 5′-AATTAACTCGAGCCACCATGGAAGTGACTGAAAGAGATGCAG-3′ (SEQ ID NO: 5) and 5′-AAAAAATCTAGATCAGCGCT CATCCAATTCATC-3′ (SEQ ID NO: 6). For bases 2695-4390, 5′-ATATATCTCGAGCCACCATGGATCCAGATGAATTGGATGAGCG-3′ (SEQ ID NO: 7) and 5′-TATATATCTAGACTAAGCAGCACCTCTCTC GTGATTTC-3′ (SEQ ID NO: 8). The fragments were cloned separately into the Xho I-Xba I sites of pSP73-Sph/A64.

Cloning of pSP73/Tie2/A64/Not. The forward primer 5′-TATATATCTAGAGCCACCATGGACTCTTTAGCCGGCTTAGTTC-3′ (SEQ ID NO: 9) and reverse primer 5′-TATATAGAATTCCTAGGCTGCTTCTTCCGCAGAGCAG-3′ (SEQ ID NO: 10) were used to amplify the Tie2 coding sequence from plasmid DNA. The fragment was cloned into the Xba I-EcoR I sites of pSP73/A64/Not.

Cloning of pSP73-Sph/TRP-2/A64. Total RNA was isolated from actively growing B16/F10.9 cells. Reverse transcription was primed with an anchored oligo dT primer and the TRP-2 cDNA was amplified from the first stand using the forward primer 5′-GATGGATCCAAGCTTGCCACCATGGGCCTTGTGGGATGG-3′ (SEQ ID NO: 11). and the reverse primer 5′-GTTAGATCTGCGGCCGCTAGGCTTCCTCCGTGTATC-3′ (SEQ ID NO: 12). The resulting product was digested with Bgl II and BamH I and cloned into the BamH I site of pSP73-Sph/A64.

Cloning of pGEM4Z/murineTERT/A64. The EcoR I fragment of pGRN188 (Geron Corp., Menlo Park, Calif.) was cloned into the EcoR I site of pGEM4Z/A64/Not. Linearization with Not I followed by in vitro transcription (Ambion mMessage mMachine kit, Austin, Tex.) yields a transcript containing 61 nt from of the polylinker of pGEM4Z, followed by 34 nt of 5′ UTR of mTERT, 3366 nt mTERT ORF, 36 nt of 3′ UTR of mTERT, 64 A residues, an Spe I site and a Not I half-site.

Cloning of pGEM4Z/murine actin/A64. Reverse transcription of total RNA from F10.9 cells was primed by oligo dT and carried out by PowerScript reverse transcriptase (Clontech). The forward primer 5′-TATATAAGCTTCTTTGCAGCTCCTTCGTTG-3′ (SEQ ID NO: 13) and the reverse primer 5′-TTTATGGATCCAAGCAATGCTGTCACCTTCCC-3′ (SEQ ID NO: 14) were used to amplify the actin coding sequence from the first-strand cDNA. The PCR fragment was cloned into the Hind III-BamH I sites of pGEM4Z/A64.

### Example 7

CTL Induction in vivo

Generation of CTL. Bone marrow precursor derived DC were generated and transfected with RNA as described above. Naive, syngeneic mice were immunized intravenously with 5×105 precursor-derived DC per mouse in 200 μl PBS, three times at 7-day intervals. Splenocytes were harvested 8-10 days after the final immunization and depleted of red blood cells with ammonium chloride Tris buffer. 10⁷ splenocytes were cultured with 2×10⁵ stimulator cells (DC electroporated with RNA) in 5 ml of IMDM with 10% FCS, 1 mM sodium pyruvate, 100 IU/ml penicillin, 100 μg/ml streptomycin and 5×10⁻⁵ M β-mercaptoethanol per well in a 6-well tissue culture plate. The responders were stimulated with the same antigen as used for the immunization. Cells were cultured for 5 days at 37° C. and 5% CO₂. Effectors were harvested on day 5 on Histopaque 1083 gradient prior to use in a CTL assay.

In vitro cytotoxicity assay. 5-10×10⁶ target cells were labeled with europium for 20 minutes at 4° C. 10⁴ europium-labeled targets and serial dilutions of effector cells at varying E:T ratios were incubated in 200 μl of complete RPMI 1640. The plates were centrifuged at 500 g for 3 minutes and incubated at 37° C. for 4 hours. 50 μl of the supernatant was harvested and europium release was measured by time resolved fluorescence ²¹. Specific cytotoxic activity was determined using the formula: % specific release={(experimental release−spontaneous release)/(total release−spontaneous release)}×100. Spontaneous release of the target cells was less than 25% of total release by detergent in all assays. Standard errors of the means of triplicate cultures was less than 5%.

### Example 8

CTL Activity in Response to Immunization with Angiogenesis-related Antigens

To determine whether immunization can break tolerance against angiogenesis-associated products, C57BL/6 mice were immunized with VEGFR-2 or VEGF mRNA-transfected syngeneic DC and CTL responses were measured in the splenocytic population following in vitro stimulation as described above. Targets used for CTL detection were syngeneic BLK.SV tumor cells (H-2^{b}) transfected with actin mRNA, VEGF mRNA or VEGFR-2 mRNA. BLK.SV cells, like most tumor cells, express VEGF as determined by RT-PCR (data not shown). As shown in FIG. 3, immunization of mice with VEGF mRNA transfected DC stimulated CTL, which recognized all BLK.SV targets. FIG. 3 demonstrates that only targets transfected with VEGFR-2 mRNA were recognized by CTL generated from mice immunized against VEGFR-2. This is consistent with the fact that BLK.SV tumor cells do not express VEGFR-2 (data not shown). In contrast, BLK.SV cells transfected with actin mRNA or other mRNAs, were not recognized by CTL generated from mice immunized against actin. This demonstrates that it is possible to break tolerance against VEGF or VEGFR-2, but not actin, despite the fact that they represent normal gene products. Presumably, this is due to the fact that VEGF and VEGFR-2, as well as many other angiogenesis-associated products, exhibit a restricted tissue-specific pattern of expression.

### Example 9

Dorsal Skin-Fold Window Chamber Assay

Details of the design and surgical technique used for the mouse dorsal skin-fold window chamber assay are described elsewhere ^{22,23}. Briefly, mice immunized with DC transfected with VEGF or VEGFR-2 or PBS were randomly divided into three groups. An investigator who was unaware of the experimental details carried out all remaining procedures and measurements. 5 days following the surgery for placing the window chambers, the mice were then implanted with tumor cells (B16/F10.9 cells expressing GFP). This approach ensured that there would not be any interference in interpretation from the vascular changes caused by surgery. Starting on day 4 post-tumor implantation the mice were evaluated for the effect of immunization on tumor growth and vascularization. Tumor areas were measured with low magnification images of the whole tumor. Tumor vasculature was evaluated based on four random tumor areas, using higher magnification (objective, ×20). Image analysis software was used to measure the cumulative length of all vessels in focus in each image. The vascular length density was calculated by dividing the total vessel length density in the frame by the area of the frame. All images were calibrated against micrometer images at the same magnification.

### Example 10

Measurement of Neoangiogenesis Using a Skin Flap Window Chamber Model

To determine whether angiogenesis is inhibited in mice immunized against either VEGFR-2 or VEGF, the development of neovasculature in a small tumor implant was followed in real time using the skin flap window chamber model described above. Mice were either injected with PBS or immunized with VEGFR-2 or VEGF mRNA-transfected DC three times at weekly intervals. 4 weeks following the last immunization a window chamber was surgically implanted. 5 days later, B16/F10.9 melanoma cells expressing green fluorescent protein (GFP) (to facilitate subsequent analysis) were implanted into the window chamber. Invasion of blood vessels into the tumor area was monitored daily and quantitated by image analysis as previously described ²³. FIG. 4 shows the invasion of blood vessels into the implanted GFP expressing (green—second and fourth columns in FIG. 4) tumor mass. Mice injected with PBS exhibit a typical pattern of microvessel invasion into the implanted tumor, illustrative of normal angiogenesis. By contrast, a significant paucity of microvasculature was seen in the implanted tumors of mice immunized against either VEGFR-2 or VEGF. This illustrates that immunization against these antigens was associated with a partial inhibition of angiogenesis. The difference between control mice injected with PBS and mice immunized against the angiogenic products was confirmed using image analysis measuring time to microvessel invasion and microvasculature density (data not shown). The data shown in FIG. 4 are representative of each group and of observations taken over time.

### Example 11

Immunization Against Endothelial Products and Tumor Antigens is Synergistic

To determine whether the reduced rate of angiogenesis that was seen in mice immunized against angiogenesis-associated product affects tumor progression, inhibition of tumor growth in mice immunized against VEGFR-2, Tie2 or VEGF was tested in the B16/F10.9 melanoma experimental metastasis model ¹⁶ and the subcutaneously implanted MBT-2 bladder tumor model ^{11,17}. RT-PCR analysis confirmed that VEGF was expressed in both B16/F10.9 and MBT-2 tumor cells whereas neither VEGFR-2 nor Tie2 were expressed in either tumor (data not shown). In the experiment shown in FIG. 5A, the B16/F10.9 experimental metastasis model was used to measure the impact of immunization on lung metastasis. The mRNAs corresponding to VEGF, VEGFR-2 or Tie2 were transfected into syngeneic bone marrow-derived DC and used to immunize C57BL/6 mice three times at weekly intervals. 8 days following the last immunization, mice were challenged intravenously with B16/F10.9 tumor cells and lung metastasis was determined 35 days later. Mice injected with PBS or immunized with DC transfected with murine actin MRNA were used as controls. As previously seen in this experimental system, immunization with B16/F10.9 tumor RNA-transfected DC inhibited the development of lung metastasis (FIG. 5A). Immunization with VEGFR-2 mRNA-transfected DC had a comparable anti-metastatic effect. On the other hand, the impact of immunization with either Tie2 or VEGF mRNA transfected DC was more pronounced. A similar pattern of tumor inhibition was seen in the MBT-2 bladder tumor model (FIG. 5B). Since VEGFR-2 or Tie2 are expressed in proliferating endothelial cells and are not expressed in either MBT-2 or B16/F10.9 tumor cells, yet tumor growth is inhibited in mice immunized against each product, the observed inhibition of tumor growth must have been mediated via inhibition of tumor angiogenesis. This conclusion is supported by the observation that immunization against VEGFR-2 is accompanied by a reduced state of angiogenesis in the immunized animal.

### Example 12

Combination Anti-angiogenic and Immunotherapeutic Treatments

To determine whether targeting the tumor for immunological destruction and simultaneously preventing tumor vasculature formation will exert a synergistic antitumor effect, B16/F10.9 and MBT-2 tumor RNA-transfected DC were used to stimulate an immune response directed against antigens expressed by the tumor cells. The source of tumor RNA was tissue cultured tumor cell lines devoid of normal cells such as endothelial cells. It also should be noted that the immune response elicited in mice immunized with tumor RNA-transfected DC is directed to unique, and not shared, tumor antigens as judged by the fact that no crossreactivity between the tumors has been observed ¹¹. FIG. 6A shows that in the B16/F10.9 tumor model, co-immunization with B16/F10.9 tumor RNA and Tie2 mRNA is superior to immunization with either RNA alone. Similarly, FIGS. 6B and 6C show that in the MBT-2 model co-immunization with MBT-2 RNA and VEGFR-2 mRNA-transfected DC was superior to using either antigen alone, leading to a significant delay in tumor onset. These experiments demonstrate the value of combined immunization against tumor and its vasculature. The polypeptide component of telomerase (TERT), which is silent in normal tissues but reactivated in over 85% of cancers ⁴³, can serve as a broadly useful antigen in cancer vaccination ^{11,44,45}. It has previously been shown that immunization against TERT can elicit CTL and protective tumor immunity against several tumors of unrelated origin ¹¹. FIG. 6B further demonstrates that immunization of mice against both VEGF and TERT is superior to immunization against either VEGF or TERT alone suggesting that targeting the two broadly expressed prototype “universal” tumor antigens could improve the efficacy of antitumor vaccination. However, as noted above, since VEGF is also expressed by tumor cells, including the B16/F10.9 and MBT-2 tumor cells used in this study, it is not clear whether the antitumor effects of immunizing against VEGF reflect a direct effect on the tumor, its vasculature, or both.

### Example 13

Immunotherapy for Pre-existing Disease

B16/F10.9 melanoma model: Mice were challenged with 1×10⁴ F10.9 cells subcutaneously (in the flank). 3 days post-tumor implantation mice were immunized intravenously with 5×10⁵ precursor-derived DC per mouse in 200 μl PBS, three times at 7-day intervals. Tumor growth was evaluated every other day starting on day 10. Mice were sacrificed once the tumor size reached 20 mm.

For experiments testing synergy between different antigens, mice were immunized with 3×105 DC in 100 μl for each antigen for a combined 6×10⁵ DC in 200 μl per mouse.

### Example 14

Impact of Anti-angiogenic and Anti-tumor Therapy on Pre-existing Disease

To determine the impact of antitumor and anti-angiogenic immunotherapy in the setting of pre-existing disease, mice were first implanted with B16/F10.9 tumor cells followed by the immunization protocol starting three days post-tumor implantation as described above. FIG. 7A shows that in this setting, the effect of anti-VEGF immunotherapy was more pronounced than immunotherapy against TERT or VEGFR-2. Co-immunizing the mice against TERT and VEGFR-2 or VEGF and VEGFR-2 was synergistic, exhibiting an enhanced antitumor effect. FIGS. 7B and 7C further demonstrate that co-immunization against another tumor-expressed antigen, TRP-2, a dominant antigen in B16 melanoma ⁴⁶, and VEGF or VEGFR-2 is synergistic, leading to a significant delay in tumor growth.

### Example 15

Effect of Anti-angiogenic Therapy on Fertility

In two previous studies, mice immunized 10 days following immunization with VEGFR-2 protein loaded DC failed to become pregnant ⁹, whereas mice immunized with an attenuated Salmonella vector encoding a VEGFR-2 cDNA exhibited a slight delay in wound healing but no impact on fertility ¹⁰.

To determine whether the anti-tumor immunotherapies of the present invention have an effect on fertility, mice were immunized with DC electroporated with VEGF, VEGFR-2 or actin RNA three times at weekly intervals. 1 week and 8 weeks after the final immunization mice were mated with non-immunized male mice. This was done in triads (2 females to a male per cage). Number of pups delivered was recorded and the pups were examined for signs of sickness and abnormality and their weight post-weaning recorded.

In this study, despite reduced rate of angiogenesis seen in mice immunized against VEGFR-2 or VEGF no signs of morbidity or mortality were seen over an extended period of observation exceeding 6 months. However, a significant albeit transient impact on fertility of mice vaccinated against VEGFR-2, but not VEGF, was noted. As shown in FIG. 8, mice vaccinated against VEGFR-2 and mated one week later failed to become pregnant whereas if mating was delayed for 8 weeks the VEGFR-2 immunized mice were fertile with litter sizes and average weight of offsprings comparable to non-immunized mice. These observations suggest that vaccination against angiogenesis-associated products can have transient adverse effects, presumably reflecting the limited persistence of an active anti-vascular immune response. The reason for the differential effects of anti-VEGF and VEGFR-2 immunization on fertility (FIG. 8A), despite a comparable inhibitory effect on angiogenesis (FIG. 4), is unclear and will require additional studies. This suggests that in the setting of immunotherapy angiogenesis-associated products/antigens will exhibit a differential toxicity profile and that it may be possible to identify angiogenic targets which exhibit significant antitumor activity yet low toxicity. Example 16

Statistics

The different experimental groups within the study were compared using the Kruskal-Wallis test. The Mann-Whitney U-test was used to determine significance in differences in lung weights between two groups. A probability of less than 0.05 (P<0.05) was used for statistical significance. To determine the significance of combination therapy between tumor antigen and angiogenesis-related antigens we determined time to tumor onset (appearance of palpable tumors) for the various groups. Comparison between two groups were done using the log-rank test (Mantel-Haenszel test). Additional comparisons between two groups were done by determining the median time to tumor onset for each group.

All documents cited above are hereby incorporated in their entirety by reference. Also incorporated by reference are the following: Plum et al, Vaccine 19:1294 (2001), Niethammer et al, Proc. Am. Ass. Can. Res. 43:324 (2002), Li et al, J. Exp. Med. 195:1575 (2002), and Wei et al, Nat. Med. 6 (10):1160 (2001).

### REFERENCES

1. Bailar J C, 3rd, Gornik H L. Cancer undefeated. N Engl J Med. 1997;336:1569-1574.

2. Gilboa E. The makings of a tumor rejection antigen. Immunity. 1999; 11:263-270.

3. Marincola F M, Jaffee E M, Hicklin D J, Ferrone S. Escape of human solid tumors from T-cell recognition: molecular mechanisms and functional significance. Adv Immunol. 2000;74:181-273

4. Rosenberg S A. Progress in human tumour immunology and immunotherapy. Nature. 2001;411:380-384.

5. Pardoll D M. Cancer vaccines. Nat Med. 1998;4:525-531.

6. Folkman J. Tumor angiogenesis: therapeutic implications. N Engl J Med. 1971;285:1182-1186.

7. Hanahan D, Folkman J. Patterns and emerging mechanisms of the angiogenic switch during tumorigenesis. Cell. 1996;86:353-364.

8. Wei Y Q, Wang Q R, Zhao X, Yang L, Tian L, Lu Y, Kang B, Lu C J, Huang M J, Lou Y Y, Xiao F, He Q M, Shu J M, Xie X J, Mao Y Q, Lei S, Luo F, Zhou L Q, Liu C E, Zhou H, Jiang Y, Peng F, Yuan L P, Li Q, Wu Y, Liu J Y. Immunotherapy of tumors with xenogeneic endothelial cells as a vaccine. Nat Med. 2000;6:1160-1166.

9. Li Y, Wang M N, Li H, King K D, Bassi R, Sun H, Santiago A, Hooper A T, Bohlen P, Hicklin D J. Active Immunization Against the Vascular Endothelial Growth Factor Receptor flk1 Inhibits Tumor Angiogenesis and Metastasis. J Exp Med. 2002;195:1575-1584.

10. Niethammer A G, Xiang R, Becker J C, Wodrich H, Pertl U, Karsten G, Eliceiri B P, Reisfeld R A. A DNA vaccine against VEGF receptor 2 prevents effective angiogenesis and inhibits tumor growth. Nat Med. 2002;8:1369-1375

11. Nair S K, Heiser A, Boczkowski D, Majumdar A, Naoe M, Lebkowski J S, Viweg J, Gilboa E. Induction of cytotoxic T cell responses and tumor immunity against unrelated tumors using telomerase reverse transcriptase RNA transfected dendritic cells. Nature Medicine. 2000;6

12. Boczkowski D, Nair S K, Snyder D, Gilboa E. Dendritic cells pulsed with RNA are potent antigen-presenting cells in vitro and in vivo. J Exp Med. 1996; 184:465-472

13. Nair S K, Boczkowski D, Morse M, Cumming R I, Lyerly H K, Gilboa E. Induction of primary carcinoembryonic antigen (CEA)-specific cytotoxic T lymphocytes in vitro using human dendritic cells transfected with RNA. Nat Biotechnol. 1998;16:364-369

14. Heiser A, Dahm P, D RY, Maurice M A, Boczkowski D, Nair S K, Gilboa E, Vieweg J. Human dendritic cells transfected with RNA encoding prostate-specific antigen stimulate prostate-specific CTL responses in vitro. J Immunol. 2000; 164:5508-5514

15. Nair S, Boczkowski D. RNA-transfected dendritic cells. Expert Rev. Vaccines. 2002;1:89-95

16. Porgador A, Tzehoval E, Vadai E, Feldman M, Eisenbach L. Combined vaccination with major histocompatibility class I and interleukin 2 gene-transduced melanoma cells synergizes the cure of postsurgical established lung metastases. Cancer Res. 1995;55:49414949.

17. Soloway M S, deKernion J B, Rose D, Persky L. Effect of chemotherapeutic agents on bladder cancer: a new animal model. Surg Forum. 1973;24:542-544

18. Inaba K, Inaba M, Romani N, Aya H, Deguchi M, Ikehara S, Muramatsu S, Steinman R M. Generation of large numbers of dendritic cells from mouse bone marrow cultures supplemented with granulocyte/macrophage colony-stimulating factor. J Exp Med. 1992; 176:1693-1702.

19. Saeboe-Larssen S, Fossberg E, Gaudernack G. mRNA-based electrotransfection of human dendritic cells and induction of cytotoxic T lymphocyte responses against the telomerase catalytic subunit (hTERT). J Immunol Methods. 2002;259:191-203.

20. Van Tendeloo V F, Ponsaerts P, Lardon F, Nijs G, Lenjou M, Van Broeckhoven C, Van Bockstaele D R, Berneman Z N. Highly efficient gene delivery by mRNA electroporation in human hematopoietic cells: superiority to lipofection and passive pulsing of mRNA and to electroporation of plasmid cDNA for tumor antigen loading of dendritic cells. Blood. 2001;98:49-56.

21. Volgmann T, Klein-Struckmeier A, Mohr H. A fluorescence-based assay for quantitation of lymphokine-activated killer cell activity. J Immunol Methods. 1989;1 19:45-51

22. Huang Q, Shan S, Braun R D, Lanzen J, Anyrhambatla G, Kong G, Borelli M, Corry P, Dewhirst M W, Li C Y. Noninvasive visualization of tumors in rodent dorsal skin window chambers. Nat Biotechnol. 1999;17:1033-1035.

java/Propub/biotech/nbt1099_{—}1033.fulltext

java/Propub/biotech/nbt1099_{—}1033.abstract

23. Li C Y, Shan S, Huang Q, Braun R D, Lanzen J, Hu K, Lin P, Dewhirst M W. Initial stages of tumor cell-induced angiogenesis: evaluation via skin window chambers in rodent models. J Natl Cancer Inst. 2000;92:143-147.

24. Veikkola T, Alitalo K. VEGFs, receptors and angiogenesis. Semin Cancer Biol. 1999;9:211-220.

25. Gale N W, Yancopoulos G D. Growth factors acting via endothelial cell-specific receptor tyrosine kinases: VEGFs, angiopoietins, and ephrins in vascular development. Genes Dev. 1999;13:1055-1066.

26. Millauer B, Longhi M P, Plate K H, Shawver L K, Risau W, Ullrich A, Strawn L M. Dominant-negative inhibition of Flk-1 suppresses the growth of many tumor types in vivo. Cancer Res. 1996;56:1615-1620.

27. Olson T A, Mohanraj D, Carson L F, Ramakrishnan S. Vascular permeability factor gene expression in normal and neoplastic human ovaries. Cancer Res. 1994;54:276-280.

28. Berkman R A, Merrill M J, Reinhold W C, Monacci W T, Saxena A, Clark W C, Robertson J T, Ali I U, Oldfield E H. Expression of the vascular permeability factor/vascular endothelial growth factor gene in central nervous system neoplasms. J Clin Invest. 1993;91:153-159.

29. Brown L F, Berse B, Jackman R W, Tognazzi K, Manseau E J, Dvorak H F, Senger D R. Increased expression of vascular permeability factor (vascular endothelial growth factor) and its receptors in kidney and bladder carcinomas. Am J Pathol. 1993;143:1255-1262.

30. Brown L F, Berse B, Jackman R W, Tognazzi K, Guidi A J, Dvorak H F, Senger D R, Connolly J L, Schnitt S J. Expression of vascular permeability factor (vascular endothelial growth factor) and its receptors in breast cancer. Hum Pathol. 1995;26:86-91.

31. Zhu Z, Witte L. Inhibition of tumor growth and metastasis by targeting tumor-associated angiogenesis with antagonists to the receptors of vascular endothelial growth factor. Invest New Drugs. 1999;17:195-212

32. Deplanque G, Harris A L. Anti-angiogenic agents: clinical trial design and therapies in development. Eur J Cancer. 2000;36:1713-1724.

33. Eatock M M, Schatzlein A, Kaye S B. Tumour vasculature as a target for anticancer therapy. Cancer Treat Rev. 2000;26:191-204.

34. Cherrington J M, Strawn L M, Shawver L K. New paradigms for the treatment of cancer: the role of anti-angiogenesis agents. Adv Cancer Res. 2000;79:1-38

35. Hanahan D. Signaling vascular morphogenesis and maintenance. Science. 1997;277:48-50.

36. Lin P, Polverini P, Dewhirst M, Shan S, Rao P S, Peters K. Inhibition of tumor angiogenesis using a soluble receptor establishes a role for Tie2 in pathologic vascular growth. J Clin Invest. 1997;100:2072-2078.

37. Lin P, Buxton J A, Acheson A, Radziejewski C, Maisonpierre P C, Yancopoulos G D, Channon K M, Hale L P, Dewhirst M W, George S E, Peters K G. Antiangiogenic gene therapy targeting the endothelium-specific receptor tyrosine kinase Tie2. Proc Natl Acad Sci U S A. 1998;95:8829-8834.

38. Siemeister G, Schirner M, Weindel K, Reusch P, Menrad A, Marme D, Martiny-Baron G. Two independent mechanisms essential for tumor angiogenesis: inhibition of human melanoma xenograft growth by interfering with either the vascular endothelial growth factor receptor pathway or the Tie-2 pathway. Cancer Res. 1999;59:3185-3191.

39. Carmeliet P, Ferreira V, Breier G, Pollefeyt S, Kieckens L, Gertsenstein M, Fahrig M, Vandenhoeck A, Harpal K, Eberhardt C, Declercq C, Pawling J, Moons L, Collen D, Risau W, Nagy A. Abnormal blood vessel development and lethality in embryos lacking a single VEGF allele. Nature. 1996;380:435-439.

40. Ferrara N, Carver-Moore K, Chen H, Dowd M, Lu L, O'Shea K S, Powell-Braxton L, Hillan K J, Moore M W. Heterozygous embryonic lethality induced by targeted inactivation of the VEGF gene. Nature. 1996;380:439-442.

41. Kim K J, Li B, Winer J, Armanini M, Gillett N, Phillips H S, Ferrara N. Inhibition of vascular endothelial growth factor-induced angiogenesis suppresses tumour growth in vivo. Nature. 1993;362:841-844.

42. Blancher C, Harris A L. The molecular basis of the hypoxia response pathway: tumour hypoxia as a therapy target. Cancer Metastasis Rev. 1998;17:187-194.

43. Shay J W, Bacchetti S. A survey of telomerase activity in human cancer. Eur J Cancer. 1997;33:787-791

44. Minev B, Hipp J, Firat H, Schmidt J D, Langlade-Demoyen P, Zanetti M. Cytotoxic T cell immunity against telomerase reverse transcriptase in humans. Proc Natl Acad Sci U S A. 2000;97:4796-4801.

45. Vonderheide R H, Hahn W C, Schultze J L, Nadler L M. The telomerase catalytic subunit is a widely expressed tumor-associated antigen recognized by cytotoxic T lymphocytes. Immunity. 1999;10:673-679.

46. Schreurs M W, Eggert A A, de Boer A J, Vissers J L, van Hall T, Offringa R, Figdor C G, Adema G J. Dendritic cells break tolerance and induce protective immunity against a melanocyte differentiation antigen in an autologous melanoma model. Cancer Res. 2000;60:6995-7001.

47. Kerbel R S. A cancer therapy resistant to resistance. Nature. 1997;390:335-336.

48. Lange-Asschenfeldt B, Velasco P, Streit M, Hawighorst T, Pike S E, Tosato G, Detmar M. The angiogenesis inhibitor vasostatin does not impair wound healing at tumor-inhibiting doses. J Invest Dermatol. 2001;117:1036-1041.

49. Quinn T E, Thurman G B, Sundell A K, Zhang M, Hellerqvist C G. CM101, a polysaccharide antitumor agent, does not inhibit wound healing in murine models. J Cancer Res Clin Oncol. 1995;121:253-256

50. Matzinger P. An innate sense of danger. Semin Immunol. 1998; 10:399-415

51. Bachmann M F, Kundig T M, Hengartner H, Zinkernagel R M. Protection against immunopathological consequences of a viral infection by activated but not resting cytotoxic T cells: T cell memory without “memory T cells”? Proc Natl Acad Sci U S A. 1997;94:640-645.

It will be understood that various details of the presently claimed subject matter can be changed without departing from the scope of the presently claimed subject matter. Furthermore, the foregoing description is for the purpose of illustration only, and not for the purpose of limitation.

What is claimed is:

1. A method for the treatment of cancer comprising administering to a patient in need thereof an immunogenic composition capable of inducing active immunity against at least one angiogenesis-related antigen.

2. The method according to claim 1, wherein said immunogenic composition comprises an angiogenesis-related antigenic polypeptide.

3. The method according to claim 1, wherein said immunogenic composition comprises a nucleic acid encoding an angiogenesis-related antigenic polypeptide.

4. The method according to claim 1, wherein said immunogenic composition comprises a plurality of antigen presenting cells presenting at least one angiogenesis-related antigen on the surface.

5. The method of claim 4, wherein said antigen presenting cells are pulsed with at least one angiogenesis-related antigen peptide.

6. The method of claim 4, wherein said antigen presenting cells are transfected with mRNA encoding at least one angiogenesis related antigen.

7. The method of claim 4, wherein said antigen presenting cells are dendritic cells.

8. The method of claim 1 wherein said angiogenesis related antigen is selected from the group consisting of Id1, Id3, VEGF, VEGFR-2, angiopoietin and Tie-2.

9. The method of claim 6, wherein said antigen presenting cells are further transfected with mRNA encoding at least one tumor antigen.

10. A composition for the treatment or prevention of cancer comprising antigen presenting cells presenting at least one angiogenesis-related antigen.

11. The composition of claim 10, wherein said angiogenesis-related antigen is selected from the group consisting of Id1, Id3, VEGF, VEGFR-2, angiopoietin and Tie-2.

12. The composition of claim 10, wherein said antigen presenting cells are dendritic cells.

13. The composition of claim 10, wherein said antigen presenting cells are transfected with mRNA encoding at least one angiogenesis-related antigen.

14. The composition of claim 10, wherein said antigen presenting cells also present at least one tumor antigen.

15. The composition of claim 14, wherein said antigen presenting cells are transfected with mRNA encoding at least one tumor antigen.

16. A method of treating cancer comprising the steps of:
i. obtaining antigen presenting cells from a cancer patient
ii. introducing into those cells in vitro, mRNA encoding an angiogenesis-related antigen and mRNA encoding a tumor antigen, thereby producing transfected antigen presenting cells and
iii. administering said transfected antigen presenting cells to said patient.

17. A method of treating cancer comprising the steps of:
i. obtaining antigen presenting cells from a cancer patient;
ii. transfecting the antigen presenting cells in vitro, with mRNA encoding an angiogenesis-related antigen and mRNA encoding a tumor antigen;
iii. contacting the transfected antigen presenting cells of step ii with T-lymphocytes to generate immune cells; and
iv. administering the immune cells to said cancer patient.

18. The method according to claim 2 wherein said immunogenic composition further comprises a tumor antigen.


SEQUENCE LISTING (14 sequences)

SEQ ID NO: 1 - DNA, 48 bp, Artificial
  Forward primer used in conjunction with SEQ ID NO 2 to amplify a region of the murine VEGF coding sequence
tatatatctagagccaccatggcacccacgacagaaggagagcagaag

SEQ ID NO: 2 - DNA, 35 bp, Artificial
  Reverse primer used in conjunction with SEQ ID NO 1 to amplify a region of the murine VEGF coding sequence
tatatagaattctcaccgccttggcttgtcacatc

SEQ ID NO: 3 - DNA, 43 bp, Artificial
  Forward primer used in conjunction with SEQ ID NO 4 to amplify a region of the murine VEGFR-2 coding sequence
tatatactcgaggccaccatggagagcaaggcgatgctagctg

SEQ ID NO: 4 - DNA, 35 bp, Artificial
  Reverse primer used in conjunction with SEQ ID NO 3 to amplify a region of the murine VEGFR-2 coding sequence
attaatctagactagttggactcaatggggccttc

SEQ ID NO: 5 - DNA, 42 bp, Artificial
  Forward primer used in conjunction with SEQ ID NO 6 to amplify a region of the murine VEGFR-2 coding sequence
aattaactcgagccaccatggaagtgactgaaagagatgcag

SEQ ID NO: 6 - DNA, 33 bp, Artificial
  Reverse primer used in conjunction with SEQ ID NO 5 to amplify a region of the murine VEGFR-2 coding sequence
aaaaaatctagatcagcgctcatccaattcatc

SEQ ID NO: 7 - DNA, 43 bp, Artificial
  Forward primer used in conjunction with SEQ ID NO 8 to amplify a region of the murine VEGFR-2 coding sequence
atatatctcgagccaccatggatccagatgaattggatgagcg

SEQ ID NO: 8 - DNA, 38 bp, Artificial
  Reverse primer used in conjunction with SEQ ID NO 7 to amplify a region of the murine VEGFR-2 coding sequence
tatatatctagactaagcagcacctctctcgtgatttc

SEQ ID NO: 9 - DNA, 43 bp, Artificial
  Forward primer used in conjunction with SEQ ID NO 10 to amplify a region of the murine Tie2 coding sequence
tatatatctagagccaccatggactctttagccggcttagttc

SEQ ID NO: 10 - DNA, 37 bp, Artificial
  Reverse primer used in conjunction with SEQ ID NO 9 to amplify a region of the murine Tie2 coding sequence
tatatagaattcctaggctgcttcttccgcagagcag

SEQ ID NO: 11 - DNA, 39 bp, Artificial
  Forward primer used in conjunction with SEQ ID NO 12 to amplify a region of the murine TRP-2 coding sequence
gatggatccaagcttgccaccatgggccttgtgggatgg

SEQ ID NO: 12 - DNA, 36 bp, Artificial
  Reverse primer used in conjunction with SEQ ID NO 11 to amplify a region of the murine TRP-2 coding sequence
gttagatctgcggccgctaggcttcctccgtgtatc

SEQ ID NO: 13 - DNA, 30 bp, Artificial
  Forward primer used in conjunction with SEQ ID NO 14 to amplify a region of the murine actin coding sequence
tatataagcttctttgcagctccttcgttg

SEQ ID NO: 14 - DNA, 32 bp, Artificial
  Reverse primer used in conjunction with SEQ ID NO 13 to amplify a region of the murine actin coding sequence
tttatggatccaagcaatgctgtcaccttccc